(19) United States
(12) Patent Application Publication
(10) Pub. No.: US 2007/0066927 A1
(43) Pub. Date: Mar. 22, 2007

(54) MEDICAL SWAB FOR STERILIZATION AND ANESTHITIZATION OF INJECTION SITES AND MINOR SURGICAL SITES

(76) Inventors: Christopher R. Akelman, Barrington, RI (US); Robert W. Treut, Providence, RI (US)

Correspondence Address: BARLOW, JOSEPHS & HOLMES, LTD., 101 DYER STREET, 5TH FLOOR, PROVIDENCE, RI, 02903, US

(21) Appl. No.: 11/534,011

(22) Filed: Sep. 21, 2006

Related U.S. Application Data
(60) Provisional application No. 60/719,145, filed on Sep. 21, 2005.

(51) Int. Cl. A61M 35/00

(52) U.S. Cl. 604/2

(57) ABSTRACT

A medical swab to sterilize and greatly cool an area of skin is disclosed. The medical swab is for medical use for pre-injection preparation, to sterilize the area of injection and reduce the pain felt by the patient. Preferably, the medical swabs are individually packaged. The swabs are saturated in isopropyl alcohol and ethyl chloride and sealed in a sterile manner. The medical swab keeps the users hand free of the saturated area of the pad so that the cooling effects of the solution are not delivered on the hand of the administrator.

### CROSS-REFERENCE TO RELATED APPLICATION

This application claims priority to earlier filed U.S. Provisional Application No. 60/719,145, filed Sep. 21, 2005, the contents of which are incorporation herein by reference.

### BACKGROUND OF THE INVENTION

1. Field of the Invention

The present invention relates generally to topical treatments to sterilize and anesthetize an injection site or minor surgical site on the skin of a patient for purposes of medical treatment and more particularly to a disposable applicator that can anesthetize and sterilize such sites.

2. Background of the Related Art

In the medical arts, patients receive injections quite frequently for any number of reasons such as vaccines to prevent illness, antibiotics to treat infections, and insulin to treat diabetes to name a few. Injections are almost always administered from a syringe with a needle that is pushed into the skin of the patient. An injection can be subcutaneous (into the fat between the skin and muscle) or intramuscular (into the muscle itself) depending on the treatment being sought. As a result, injections administered with syringes can be painful to the patient. During all injections, though, the injection site on the skin must be sterilized to prevent infection and is also frequently anesthetized for the patient's comfort. Although most injections are performed by medical professionals (such as doctors, nurses, paramedics, physicians assistants, etc.), some are self-administered by patients themselves, such as insulin shots, or even by other individuals, such as family members. As a matter of convenience and not by way of limitation, only medical professionals will be referred to in this specification, but all individuals that have a need to administer an injection of any sort are intended to be included.

Typically a medical professional sterilizes the injection site by applying a solution isopropyl alcohol with a cotton swab liberally at the injection site. The medical professional next applies an anesthetizing agent with a second cotton swab to the now sterilized injection site in a similar manner. The anesthetizing agent is frequently a solution of ethyl chloride.

Although not a true anesthetic, ethyl chloride provides an anesthetic effect at the injection site due to its high volatility. The high volatility of the ethyl chloride results in the substance evaporating quickly, thus providing a rapid evaporative cooling effect to the injection site, which numbs the area and allows the needle of a syringe to be inserted into the skin with little or no pain. The medical professional may then administer the injection to the patient with a reduced likelihood of infection and with more comfort.

In addition to the administration of injections, medical professionals frequently follow the same sterilization and anesthetization steps for procedures involving minor surgical procedures, such as the taking of biopsies, the insertion of stitches, and removal of moles to name a few.

This process, however, suffers from several disadvantages. First, the process is less efficient because the medical professional must swab the injection site twice; once to sterilize the area and a second time to anesthetize the area. Second, the process leads to excess waste because two cotton swabs must be expended for every single procedure. Medical waste is costly to dispose of and it is desirable to reduce the amount generated. Third, the amount of anesthetizing agent and sterilizing agent applied is variable due to the fact that each medical professional will apply a different amount of each agent. In the best case, excess agents are applied causing waste of the agents. In the worst case, too little of the agents are being applied defeating the purpose of applying them in the first place. Ineffective amounts of the anesthetizing agent can lead to pain and discomfort during the procedure and ineffective amounts of the sterilizing agent can lead to an infection. Therefore, there is a need to standardize the amounts of the sterilizing agent and anesthetizing agent applied, reduce the waste created and increase the efficiency and convenience for providing injections and minor surgical procedures to patients.

### SUMMARY OF THE INVENTION

The present invention solves the problems of the prior art by providing a disposable swab that can be used to administer a sterilizing agent and an anesthetizing agent to an injection site or minor surgical site on a patient in a single application.

The medical swab is intended to both sterilize and greatly cool an area of skin. The application of this invention will be in medical use, for pre-injection preparation, to sterilize the area of injection and reduce the pain felt by the patient.

Individually packaged pads will be made, saturated in isopropyl alcohol and ethyl chloride and sealed in a sterile manner. The design of the pad (pictured in attached drawings) will aim to keep the users hand free of the saturated area of the pad so that its cooling effects will not be delivered on the hand of the administrator.

### BRIEF DESCRIPTION OF THE DRAWINGS

These and other features, aspects, and advantages of the present invention will become better understood with reference to the following description, appended claims, and accompanying drawings where:

FIG. 1. is a top view of the medical swab of the present invention; and

FIG. 2 is a side view of the medical swab of the present invention.

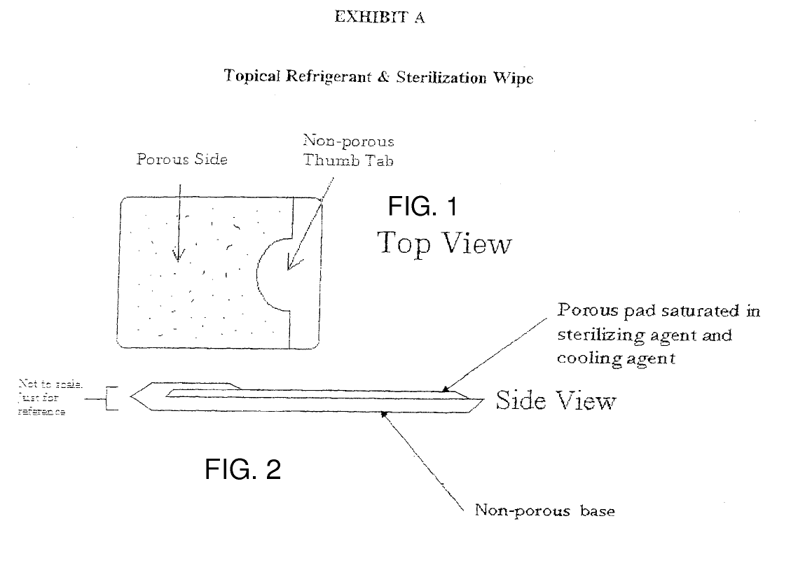

### DETAILED DESCRIPTION OF THE PREFERRED EMBODIMENT

in the figure attached as Exhibit A, the swab of the present invention includes a non-porous base having a front and back surface. A porous pad is attached to the front surface of the base and is saturated with a solution containing a sterilizing agent, such as isopropyl alcohol, and an anesthetizing agent, such as ethyl chloride.

Although isopropyl alcohol is preferred for the sterilizing agent and ethyl chloride is preferred for the anesthetizing agent, suitable substances having a similar sterilizing effect or anesthetic effect could be respectively substituted for one of the preferred agents provided that it does not react with the other agent. The desirability of isopropyl alcohol and ethyl chloride lies in the fact that the two substances will not react with each other and thus possibly injure the patient or cause other undesirable results.

The swab of the present invention includes a non-porous thumb tab extending over the front surface of the non-porous base and the porous pad. In use, this allows the medical professional to securely grip the swab between the non-porous base and non-porous thumb tab without exposing his or her digits to the anesthetizing agent retained in the saturated porous pad. The medical professional can then swab the injection site once and apply both the sterilizing agent and the anesthetizing agent to the injection site in one application.

The entire swab is enclosed in an opaque non-porous wrapper to prevent evaporation of the anesthetizing agent and the sterilizing agent, thus keeping the porous pad moist with the solution. The opaque wrapper protects the solution and swab from degradation due to exposure to light and ultraviolet radiation. Alternatively, the swab could include a tear-away strip that is attached to the non-porous-base and encloses the porous pad to seal the porous pad from the outside air and light. Also, the swabs could be enclosed in a substantially air-tight, opaque container.

Therefore, it can be seen that the present invention provides a unique solution to the problems of the prior art by providing a disposable swab that can be used to administer a sterilizing agent and an anesthetizing agent to an injection site or minor surgical site on a patient in a single application, thus greatly reducing medical waste and increasing the efficiency and convenience of administering injections and performing minor surgical procedures.

It would be appreciated by those skilled in the art that various changes and modifications can be made to the illustrated embodiments without departing from the spirit of the present invention. All such modifications and changes are intended to be within the scope of the present invention except as limited by the appended claims.

What is claimed is:

1. A medical swab comprising:
a non-porous base having a front portion and back portion;
a porous pad attached to said front portion of said non-porous base and saturated in a solution consisting essentially of
a topical sterilization agent; and
a topical anesthetization agent.

2. The medical swab of claim 1, further comprising a non-porous thumb tab extending over said front portion of said non-porous base.

3. The medical swab of claim 1, wherein the topical sterilization agent is isopropyl alcohol.

4. The medical swab of claim 1, wherein the topical anesthetization agent is ethyl chloride.
